(19) United States
(12) Patent Application Publication
(10) Pub. No.: US 2024/0307257 A1
(43) Pub. Date: Sep. 19, 2024

(54) METHOD OF MANUFACTURING AN UTENSIL COMPRISING A PLURALITY OF UTENSIL ELEMENTS

(71) Applicant: HYBRID REALITY GMBH, Berlin (DE)

(72) Inventors: Ani MANERA DA SILVA, Amsterdam (NL); Cord LABUHN, Berlin (DE)

(73) Assignee: HYBRID REALITY GMBH, Berlin (DE)

(21) Appl. No.: 18/573,458

(22) PCT Filed: Jun. 20, 2022
(86) PCT No.: PCT/EP2022/066694

§ 371 Date: Dec. 22, 2023

(30) Foreign Application Priority Data
Jun. 23, 2021 (DE) 10 2021 116 255.6

(51) Int. Cl. A61H 19/00; A41D 19/00; D04H 1/4258

(52) U.S. Cl. CPC A61H 19/32; A41D 19/0068; D04H 1/4258

(57) ABSTRACT

The invention relates to a method for producing a utensil comprising a plurality of utensil elements with the method steps of providing a first utensil element, wherein the first utensil element was produced from a hydrogel, providing a second utensil element, providing a connecting element and positively and/or non-positively connecting the first utensil element to the second utensil element with the aid of the connecting element, and a nonwoven made from a hydrogel of bacterial nanocellulose which is loaded with a sliding agent, and the use of a hydrogel of bacterial nanocellulose as a stimulation utensil.

The invention relates to a method for producing an utensil comprising a plurality of utensil elements with the method steps of providing a first utensil element, the first utensil element being produced from a hydrogel, providing a second utensil element, providing a connecting element and form-fitting and/or force-fitting connection of the first utensil element to the second utensil element with the aid of the connecting element. Furthermore, a nonwoven made of a hydrogel of bacterial nanocellulose, which is loaded with a sliding agent, as well as the use of a hydrogel of bacterial nanocellulose as a stimulation utensil is an object of the invention.

Dimensionally stable hydrogels made from bacterial nanocellulose are known. These are produced by cultivating a suitable bacterial strain in an aqueous and acid-buffered nutrient medium, whereby a dimensionally stable hydrogel forms at the interface between the nutrient medium and the air during the cultivation process, which can sometimes last several weeks. Depending on the manufacturing process, hydrogels produced in this way also meet the requirements for vegan products. In the following, these dimensionally stable hydrogels are also referred to as “nanocellulose gels” or “BNC nonwovens”.

Nanocellulose gels can be used as massage sponges, washcloths, wet wipes or protective films due to their structural similarity to human skin, their good compatibility with the human organism and their high water retention capacity. Nanocellulose gels can be loaded and equipped with special properties (such as color, taste, fragrance, surface structure, permeability, active ingredient loading). In-situ modification, i.e. influencing the synthesis during the cultivation process by adding different additives to the culture medium, is also known. It is also known to modify biomaterials based on bacterial nanocellulose after it has been synthesized (post-modification).

‘Microbial polymers’ include polymers produced by a microorganism, such as bacteria, fungi or algae. Nanocellulose is preferably synthesized by cultivating microbial strains such as Gluconacetobacter, Enterobacter, Agrobacterium, Pseudomonas, and Rhizpobium. In addition to Gluctonacetobacter hansenii and Gluconacetobacter kombuchae, the bacterial strain Gluconacetobacter xylinus, which has also been most extensively researched and documented in this context, is particularly suitable for the production of nanocellulose gels. The nanocellulose gel is produced by microorganisms at the interface between air and a nutrient medium containing D-glucose. In the alternative production processes discussed below, sucrose in aqueous solution is the carbon source. The bacteria expel the cellulose as fibrils, which assemble into fibers at the interface between the nutrient medium and the air. The result is a three-dimensionally interwoven fiber network consisting of about 99% water and 1% nanocellulose.

Due to the special material properties of this biopolymer, its extremely high biocompatibility, structural similarity to the body's own protein-based tissue, variety of shapes and numerous modification options, it is already being used in pharmaceuticals, medicine, cosmetics and food chemistry. Bacterial nanocellulose can be sterilized under normal conditions and is characterized by a high water content and mechanical stability, while its surface and consistency are described as pleasantly soft and particularly smooth. In cosmetics, for example, it appears enriched with active ingredients and vitamins in the form of face masks. In medicine, blood vessels, implants and wound dressings made from bacterial nanocellulose are being researched and used.

Sources:

K.-Y. Lee, J. J. Blaker, A. Bismarck: Surface functionalization of bacterial cellulose as the route to produce green polylactide nanocomposites with improved properties, Composites Science and Technology (2009),

D. Klemm, D. Schumann, F. Kramer, N. HeBler, M. Hornung, H.-P. Schmauder, S. Marsch: Nanocelluloses as Innovative Polymers in Research and Application. Advances in Polymer Science (2006), 205(Polysaccharides II).

H. Wang, F. Guan, X. Ma, S. Ren: Production and performance determination of modified bacterial cellulose, Shipin Keji (2009), (5), 28-31;

N. Hessler, D. Klemm: Alteration of bacterial nanocellulose structure by in situ modification using polyethylene glycol and carbohydrate additives, Cellulose (Dordrecht, Netherlands) (2009), 16(5), 899-910;

D. Klemm, D. Schumann, F. Kramer, N. HeBler, M. Hornung, H.-P. Schmauder, S. Marsch: Nanocelluloses as Innovative Polymers in Research and Application. Advances in Polymer Science (2006), 205(Polysaccharides II), 49-96;

M. Seifert: Modification of the structure of bacterial cellulose by the composition of the culture medium in the cultivation of Acetobacter xylinum.

Nanocellulose gels according to the invention in the form of purified nonwovens can be used for massage purposes, are used in physiotherapy, healing practices, osteopathy, body therapy (also as cooling or heating pads, massage aids, haptic stimulants) as well as in body hygiene (wet wipes, refreshing wipes, probiotic washcloths), for disinfection (loaded with disinfectants) as a stimulation aid or protective film and actively for medical purposes. In addition, a nanocellulose nonwoven can be used as a massage glove, washcloth or a means of applying cosmetic products (lotions, creams, oils) over large areas. There is an increased demand for nanocellulose gels that can also, but not exclusively, be individually loaded by a user after the gels have been synthesized. An unloaded nanocellulose gel is required for this post-modification. The synthesized hydrogel must be as free as possible from bacterial residues that may adhere during the synthesis of the gel or be retained in the fiber network. Other contaminants, particularly those that are harmful to health, must also be at least below a limit value. At the same time, it must be taken into account during the cleaning process that the hydrogel is an organic material, so the cleaning process must be correspondingly gentle in order to maintain the desired properties. Utensils made of artificial polymers are used in a wide variety of forms for stimulation (vibrators, dildos, masturbators, dolls, vaginal prostheses for transsexuals, penis rings, plastic tubes that can be filled with water, etc.) and are produced industrially by the millions, rarely finding their way back into a recycling cycle or offering few options for modifying functions.

It is therefore the task of the present invention to provide a method for producing a utensil which makes it possible to produce different utensils with which it is possible to stimulate different parts of the body. At the same time, the method should be quick and easy to carry out as well as inexpensive and environmentally friendly.

It is also the task of the present invention to provide a nonwoven made of a hydrogel of bacterial nanocellulose, with which different utensils for stimulating different parts of the body can be combined and which is largely free of foreign substances and can be produced quickly, inexpensively and in an environmentally friendly manner.

The problem is solved by means of the method for manufacturing a utensil comprising a plurality of utensil elements according to claim 1. Advantageous embodiments of the invention are set out in the sub-claims.

The process for manufacturing a utensil has four process steps: In the first process step, a first utensil element is provided. The first utensil element is made from a hydrogel. In the second process step, a second utensil element is provided. The second utensil element can also be made of a hydrogel. In the context of this document, dimensionally stable hydrogels made of bacterial nanocellulose are also referred to as “nanocellulose gels” or “BNC nonwovens” and are used synonymously.

However, other embodiments are also possible in which the second utensil element is made, for example, from a fabric, a plastic, a metal or from a combination of the aforementioned materials. In the third process step, a connecting element is provided. In the fourth method step, a positive and/or non-positive connection is produced between the first utensil element and the second utensil element by means of the connecting element.

Form-fit connections are created by the interlocking of at least two connecting partners. This means that the connecting partners cannot come loose, even without or with interrupted force transmission. Pin-type connecting elements also include rivets and screws, whereby screw connections are usually both form-fit and force-fit. Force-fit connections require a normal force on the surfaces to be connected. Their mutual displacement is prevented as long as the counterforce caused by the static friction is not exceeded. Massage tools, bags, containers such as jars, bottles or tubes can be produced using the positive and non-positive connection. These can be combined in a shaping and stabilizing way or used for storage.

With the right design, the utensil can also intensify haptic and visual perception, be used as a toy, haptic stimulant and sliding agent or provide protection against mechanical overstress.

Using cutting and milling tools (knives, scissors, punches, lasers), 3D printing processes or computer-aided fermentation, shapes can be modeled from a solidly grown BNC block or a membrane of varying thickness that would not be created by purely organic growth in stationary cultivation. A solid BNC cylinder shape, for example, can be drilled or milled in the middle to insert a stabilizing element and give the utensil stability and sturdiness. After cultivation, BNC fiber layers can be removed by laser under computer control to create new surface structures or three-dimensional bodies with new properties, such as modified frictional resistance or for shaping new types of exoprostheses. Pressing and punching tools can also be used to modify the surface structure.

In an advantageous embodiment of the invention, the connecting element is a yarn, a ring, a clip, a rubber, a clip and/or a rivet. The connecting element can be selected to suit the intended use.

In a further embodiment of the invention, a plurality of connecting elements are used to connect the first and second utensil elements. A combination of several connecting elements produces a particularly durable connection between the utensil elements that is suitable for the intended use.

In a further embodiment of the invention, different connecting elements are used to connect the first and second utensil elements. A particularly durable connection between the utensil elements that is suitable for the intended use is produced by a combination of several connecting elements, including different ones.

In an advantageous embodiment of the invention, the utensil elements produced from a hydrogel are produced by a process comprising the process steps of providing and inoculating the sugar-containing solution with a bacterial strain and cultivating the solution. In the first process step, a sugar-containing solution is provided. Fructose or sucrose can serve as the carbon source, in the simplest case glucose in an aqueous and acid-buffered culture medium, whereby crystalline D-glucose is dissolved in water with, for example, sodium hydrogen phosphate and citric acid at a concentration of 2% to 20% by weight. This results in a buffered pH value in the slightly acidic range. The sugar-containing solution is then inoculated with a bacterial strain. Nanocellulose gels are preferably synthesized using microbial strains such as Gluconacetobacter, Enterobacter, Agrobacterium, Pseudomonas and Rhizpobium. The bacterial strain Gluconacetobacter xylinus, also known as Komagataeibacter xylinus, is particularly suitable for the production of nanocellulose gels. Other possible bacterial strains are Gluconacetobacter kombuchae, Komagataeibacter hansenii, Gluconobacter oxydans, Saccharomyces ludwigii, Saccharomyces apiculatus and Saccharomyces cerevisiae. In the next process step, the solution is cultivated, i.e. conditions are created and maintained that ensure metabolization of the nutrients by the bacteria. Cultivation takes place over a period of 2 to 25 days. The dimensionally stable hydrogel is produced by microorganisms at the interface between air and the nutrient medium. The bacteria expel the cellulose as fibrils, which aggregate to form fibers at the interface between the culture medium and the air. This creates a three-dimensionally interwoven fiber network consisting of around 99% water and 1% nanocellulose. In the final step of the process, the hydrogel created by the cultivation is washed to achieve a level of purity that is safe for health.

In a further development of the invention, the hydrogel resulting from the cultivation is washed. During the cultivation of bacterial nanocellulose, impurities occur depending on the production process with different culture media and bacterial strains, which are taken into account with a process step of purification after cultivation. Such a process step is necessary in order to use the bacterial nanocellulose as a nonwoven, material, designed object or loadable carrier for use on the skin.

In an advantageous embodiment of the invention, the hydrogel resulting from the cultivation is washed in an alkaline solution containing 5 wt. % to 50 wt. % at 37° C. to 142° C. for 5 min to 400 min, wherein the alkaline solution is a 5 wt. % to 50 wt. % sodium hydroxide solution. Preferably, the temperature interval for the cleaning process is 90° C. to 142° C., particularly preferably 100° C. to 142° C. The duration of the washing process is preferably 60 min to 400 min, particularly preferably 120 min to 400 min. The hydrogels produced by this process according to the invention are very easy to wash off due to their surface structure and, particularly in their synthesized pure form, are insensitive to cleaning agents which are also used on the skin (soap, washing-up liquid etc.). The hydrogels can be boiled in water or sterilized with hot steam or an electron beam and can also be cleaned in the dishwasher without deformation. They are therefore reusable and the hydrogels are completely biodegradable when disposed of properly.

In an advantageous embodiment of the invention, the washing process is followed by rinsing with distilled water for 30-120 minutes at 80° C., if necessary several times, and sterilization of the hydrogel. The cleaning process can optionally be concluded with sterilization in order to kill other microorganisms and ensure the longest possible shelf life in the packaging. Sterilization can be carried out in an autoclave or using the e-beam method. Electron beam sterilization (e-beam process) is based on the penetration of the products to be sterilized, including the packaging, by high-energy electrons, which have an ionizing and therefore germ-killing effect. The sterilization of medical articles by e-beam is one of the most efficient and safest processes, as not only all forms of bacteria, but also viruses, spores and NDA fragments are rendered biologically ineffective. Washing with sodium hydroxide solution in a concentration and duration adapted to the strength and composition of the hydrogel makes the hydrogel more flexible, softer and more pliable in its cell structure without significantly reducing its stability and water retention capacity for its intended purpose. NaOH can be used in varying concentrations (0.1 molar to 1 molar) to inactivate the bacterial strains and to wash out endotoxins and other foreign substances from the culture medium during fermentation. Sterilization by boiling and sterilization by steam pressure or e-beam can also be used to significantly extend the life cycle of the resistant hydrogels in their final product form and prepare them for reuse. Active ingredient-laden or contaminated hydrogels can thus be prepared for reuse on the skin and can be reused many times if used properly. This way, deposits and unwanted bacteria can be washed out to ensure that the hydrogel is free of germs and impurities before reuse. The pH value of a hydrogel can thus be influenced and adjusted to a desirable range for the skin.

In a further embodiment of the invention, the utensil is a stimulation utensil. The utensil is used, for example, to hydrate or lubricate the skin, to cool or warm certain parts of the body, to supply the skin with active ingredients and to protect the skin from unwanted contact, for example with bacteria, viruses, pollutants, microplastics or dermatologically questionable materials. Objects and surfaces can also be gently cleaned with a suitably prepared wet wipe made of bacterial nanocellulose (loaded with disinfectant, for example).

The problem is also solved by means of the nonwoven made of bacterial nanocellulose with a cellulose content of more than 0.5% by weight. Further advantageous embodiments of the invention are also set out in the subclaims.

The dimensionally stable hydrogel of bacterial nanocellulose according to the invention has a cellulose content of more than 0.5% by weight. The nonwoven has a fiber length of 5 μm to 15 mm, preferably the nonwoven has a fiber length of 10 μm to 10 mm, more preferably 50 μm to 5 mm and most preferably 100 μm to 1 mm. The diameter of the fibers is 60±15 nm, the density of the nonwoven is 1-1.5 g/cm³.

According to the invention, the nonwoven is loaded with a sliding agent. A sliding agent is used for inserting probes, endoscopes or ultrasound heads into the body through natural openings in order to minimize skin irritation. Examples include the insertion of gastric tubes, spiral tubes, bladder catheters, endotracheal tubes during intubation or rectoscopy. Medical sliding agents often contain a disinfectant additive, as mucous membranes in particular are very sensitive to pain, but also absorb the medication quickly. Sliding agents also serve to reduce friction on the sensitive mucous membranes of the external genital organs. Sliding agents can compensate for insufficient natural lubrication. They moisturize dry mucous membranes, thus preventing pain or are simply used to enrich lovemaking.

Sliding agents that are not water-based can damage plastic utensils or condoms used for contraception. These are usually made of natural rubber latex and can therefore react on contact with organic oils, silicones and greasy lotions, causing them to tear or at least become permeable to viruses. In the case of condoms, protection against pregnancy or sexually transmitted diseases is no longer guaranteed in such a case. The nonwoven according to the invention and a utensil made from it are resistant to such a sliding agent and are therefore safe to use.

In a further embodiment of the invention, the nonwoven has a water content of between 80% and 99.5% by weight. Thus, with about 99% by weight, a very large amount of water can be absorbed and a dimensionally stable hydrogel with a high water retention capacity can be formed. Grown under appropriate conditions, the bacterially synthesized nanocellulose thus achieves a high degree of purity, a high degree of polymerization (2000-8000) and a high degree of crystallinity (60-90%). This results in exceptional material properties such as high chemical and thermal stability, biocompatibility and high mechanical stability and tensile strength.

In a further embodiment of the invention, the sliding agent is a lubricant. The sliding agent is used to reduce friction between the nonwoven and the skin or mucous membranes of different parts of the body.

In a further embodiment of the invention, the sliding agent is an oil-containing, fat-containing, fat-free and/or silicone-containing substance. The nonwoven according to the invention is chemically resistant to the commonly used sliding agents and can therefore be loaded with a variety of sliding agents.

In a further embodiment of the invention, the sliding agent is glycerin. Glycerine is a frequently used component of sliding agents, inexpensive and readily available. At the same time, it is harmless to the organism, harmless to the skin and widely used as an ingredient in cosmetics such as creams.

In a further embodiment of the invention, the sliding agent is present in the nonwoven as a water-containing emulsion. The nonwoven made of bacterial nanocellulose can be pre-treated (inlaid) with a (3% by weight) glycerine solution at the factory or in home use and achieves a weight reduction to 3-12% of the original hydrated form by various known drying processes (such as air drying, spray drying, freeze drying, oven drying). When re-swollen in water or aqueous solution, the hydrogel absorbs up to 90% of the original amount of liquid and is able to bind this liquid thanks to its nanofiber network with high water retention capacity.

After pre-treatment, such as soaking in glycerine (1-10% in aqueous solution), gentle drying can be achieved so that the BNC can be swollen in water or additives in aqueous solution and can repeatedly assume large parts of its original mechanical properties.

In a further embodiment of the invention, the nonwoven is prepared according to one or more of claims 1 to 9. Nanocellulose is preferably synthesized by culturing microbial strains such as Gluconacetobacter, Enterobacter, Agrobacterium, Pseudomonas, and Rhizpobium. In addition to Gluctonacetobacter hansenii and Gluconacetobacter kombuchae, the bacterial strain Gluconacetobacter xylinus, which has also been most extensively researched and documented in this context, is particularly suitable for the production of dimensionally stable nanocellulose gels. The hydrogel is produced by microorganisms at the interface between air and a nutrient medium containing sugar. The bacteria expel the cellulose as fibrils, which assemble into fibers at the interface between the nutrient medium and the air. The result is a three-dimensionally interwoven fiber network consisting of about 99% water and 1% nanocellulose. From a chemical point of view, bacterially synthesized nanocellulose (BNC) is identical to plant cellulose, but differs significantly due to the specific biosynthesis process. BNC typically has a fiber diameter of around 40-60 nm, which corresponds to one hundredth of the fiber diameter of a plant cellulose or cotton fiber. Depending on the manufacturing process, fiber diameters can also range from 20-100 nm.

This creates a nanostructured network with a large specific surface area (60-100 m²/g), which allows intensive interaction with its environment. This allows a very large amount of water to be absorbed (around 99% by weight) and a dimensionally stable hydrogel with a high water retention capacity to be formed. Grown under appropriate conditions, the bacterially synthesized nanocellulose thus achieves a high degree of purity.

The task is also solved by using a hydrogel of bacterial nanocellulose as a stimulation utensil. Further advantageous embodiments of the invention are also set out in the subclaims.

According to the invention, the hydrogel made of bacterial nanocellulose is used as a stimulation utensil. The utensil is used, for example, for hydrating or lubricating the skin, cooling or warming certain parts of the body, supplying the skin with active ingredients and protecting the skin from undesired contact, for example with bacteria, viruses, pollutants, microplastics or dermatologically questionable materials.

In a further embodiment of the invention, the hydrogel is produced from bacterial nanocellulose by a process comprising the process steps of providing and inoculating the sugar-containing solution with a bacterial strain and cultivating the solution. In the first process step, a sugar-containing solution is provided. Fructose or sucrose can serve as the carbon source, in the simplest case glucose in an aqueous and acid-buffered culture medium, whereby crystalline D-glucose is dissolved in water with, for example, sodium hydrogen phosphate and citric acid at a concentration of 2% to 20% by weight. This results in a buffered pH value in the slightly acidic range. The sugar-containing solution is then inoculated with a bacterial strain. Nanocellulose gels are preferably synthesized using microbial strains such as Gluconacetobacter, Enterobacter, Agrobacterium, Pseudomonas and Rhizpobium. The bacterial strain Gluconacetobacter xylinus, also known as Komagataeibacter xylinus, is particularly suitable for the production of nanocellulose gels. Other possible bacterial strains are Gluconacetobacter kombuchae, Komagataeibacter hansenii, Gluconobacter oxydans, Saccharomyces ludwigii, Saccharomyces apiculatus and Saccharomyces cerevisiae. In the next process step, the solution is cultivated, i.e. conditions are created and maintained that ensure metabolization of the nutrients by the bacteria. Cultivation takes place over a period of 2 to 25 days. The dimensionally stable hydrogel is produced by microorganisms at the interface between air and the nutrient medium. The bacteria expel the cellulose as fibrils, which aggregate to form fibers at the interface between the culture medium and the air. This creates a three-dimensionally interwoven fiber network consisting of around 99% water and 1% nanocellulose. In the final step of the process, the hydrogel created by the cultivation is washed to achieve a level of purity that is harmless to health.

In a further development of the invention, the hydrogel is used for haptic stimulation. The hydrogel can intensify haptic and visual perception, be used as a toy, haptic stimulant and sliding agent or also provide protection against mechanical overstress.

In a further embodiment of the invention, the hydrogel is used for body and/or physiotherapy or immersive simulation. Examples include hydrating or lubricating the skin, cooling or warming certain parts of the body, supplying the skin with active ingredients and protecting the skin from unwanted contact, for example with bacteria, viruses, pollutants, microplastics or dermatologically questionable materials.

In a further development of the invention, the hydrogel is used as a stimulation utensil, washcloth, wet wipe and/or massage utensil. Already in use as a carrier system in cosmetics (active ingredient-soaked cloth masks, face pads) and in medicine (wound dressings, wound plasters) as disposable products in mostly aluminium packaging, the novel applications of bacterial nanocellulose are now aimed at reusability, the reduction of packaging waste and transport routes, customizable application scenarios (DIY cosmetics) and household (hygiene, wet wipes, disinfectant wipes, medical self-care). The use of non-allergenic, organic, biodegradable polymers as an alternative to certain latex or petroleum-based products can be made resource-saving and climate-neutral with this technology and offers the greatest possible compatibility for allergy sufferers and sensitive skin types.

In a further embodiment of the invention, the nonwoven is produced according to one or more of claims 1 to 9.

In a further embodiment of the invention, the hydrogel is a nonwoven according to one or more of claims 10 to 15.

Examples of embodiments of the method according to the invention for producing a utensil and of the nonwoven fabric according to the invention from a hydrogel are shown schematically in simplified form in the drawings and are explained in more detail in the following description.

It shows:

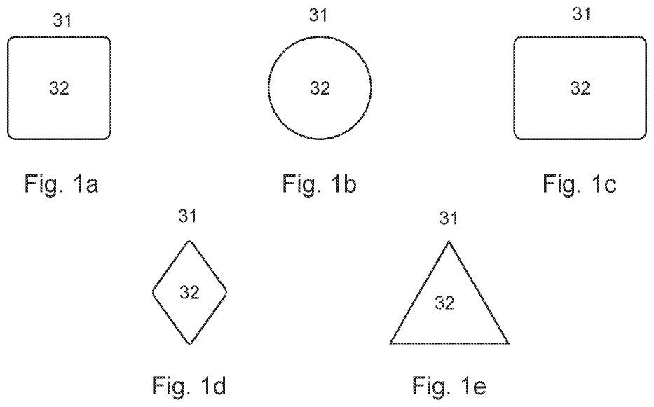

FIG. 1a: Top view of an in-situ template

FIG. 1b: Top view of another in-situ template

FIG. 1c: Top view of another in-situ template

FIG. 1d: Top view of another in-situ template

FIG. 1e: Top view of another in-situ template

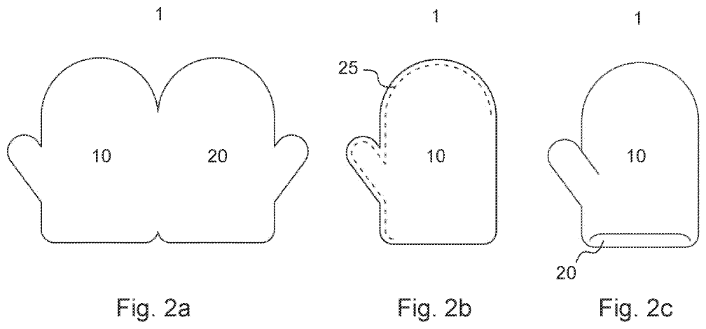

FIG. 2a: Top view of a utensil

FIG. 2b: Top view of a utensil, upper side

FIG. 2c: Top view of a utensil, underside

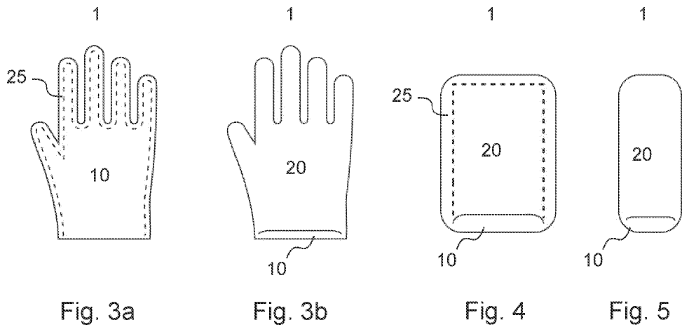

FIG. 3a: Top view of another utensil, upper side

FIG. 3b: Top view of another utensil, underside

FIG. 4 Top view of another utensil

FIG. 5: Top view of another utensil

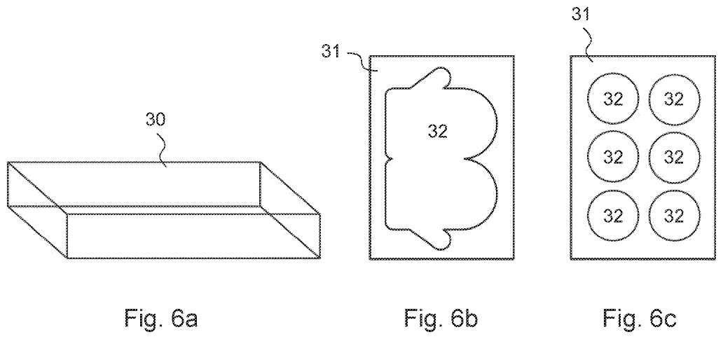

FIG. 6a: Cultivation vessel

FIG. 6b: Top view of an in-situ template

FIG. 6c: Top view of another in-situ template

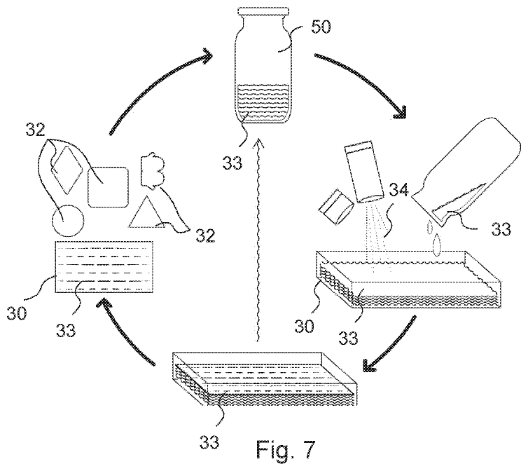

FIG. 7: Process for producing a utensil element from bacterial nanocellulose

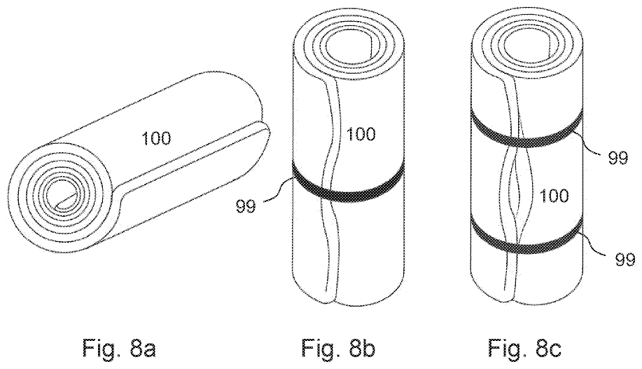

FIG. 8a: Cylindrical BNC objects

FIG. 8b: Cylindrical BNC object with a fixing element

FIG. 8c: Cylindrical BNC object with two fixing elements

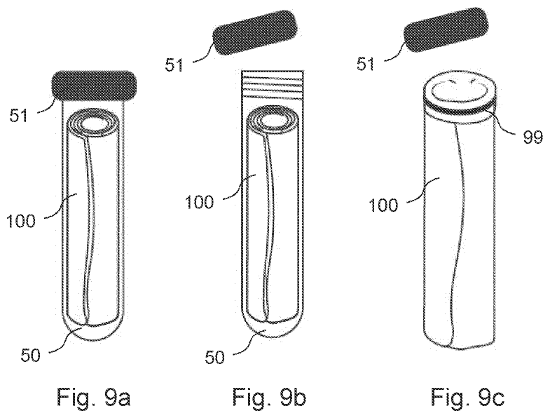

FIG. 9a: Packaging of a cylindrical BNC object, screw-top jar closed

FIG. 9b: Packaging of a cylindrical BNC object, screw-top jar opened

FIG. 9c: Connection of packaging with BNC objects as sheathing with the help of an elastic connecting element for fixation

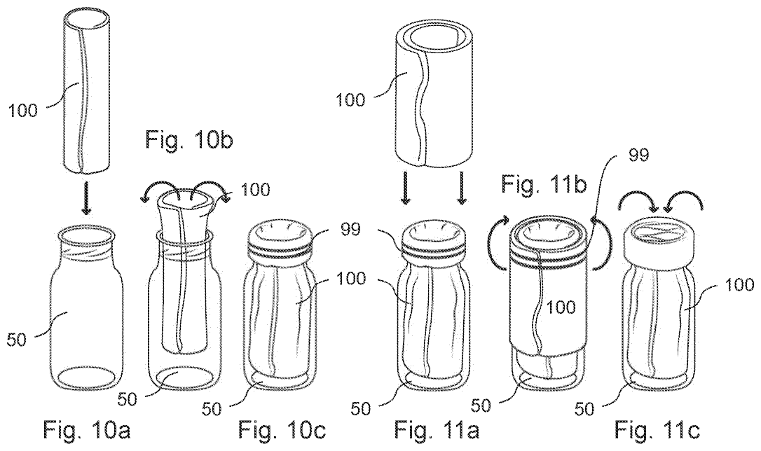

FIG. 10a: Packaging of another cylindrical BNC object, unpackaged

FIG. 10b: Combination of packaging and cylindrical BNC object

FIG. 10c: Fixing a cylindrical BNC object to the packaging

FIG. 11a: Another cylindrical BNC object, unpackaged

FIG. 11b: Combination of packaging and cylindrical BNC object, sheathing

FIG. 11c: Further cylindrical BNC object fixed to packaging, shortened

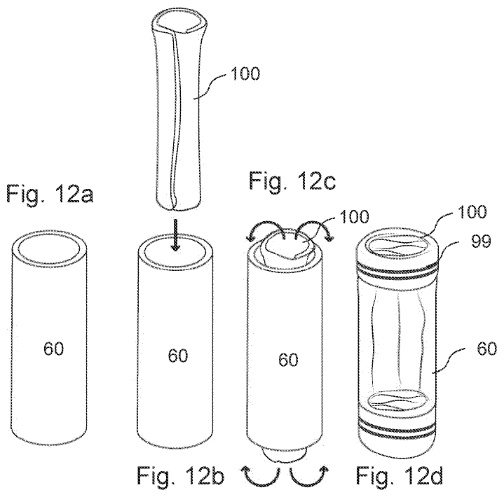

FIG. 12a: Shaping cylinder

FIG. 12b: Shaping cylinder for holding a BNC object

FIG. 12c: Shaping cylinder with BNC inlay

FIG. 12d: Shaping cylinder with fixed BNC elements

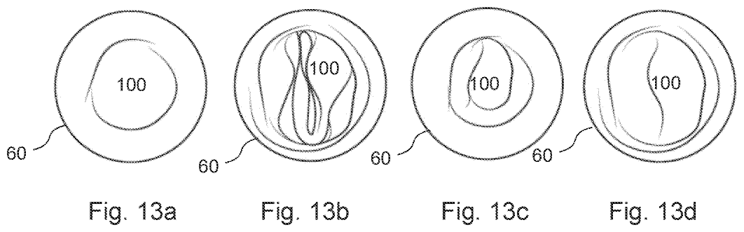

FIG. 13a: Top view of another cylindrical BNC object

FIG. 13b: Top view of another cylindrical BNC object

FIG. 13c: Top view of another cylindrical BNC object

FIG. 13d: Top view of another cylindrical BNC object

FIG. 1 shows different embodiments of in-situ templates 31 for manufacturing a utensil element 10. The templates 31 can have different shapes, here for example a square (FIG. 1a), a circular (FIG. 1b) or a rectangular (FIG. 1c), a diamond-shaped (FIG. 1d) or a triangular (FIG. 1e) shape. Other shapes are conceivable. The templates 31 have the recess 32 in which the hydrogel is cultivated. The hydrogel is produced by microorganisms at the interface between air and a nutrient medium containing sugar. The bacteria expel the cellulose as fibrils, which aggregate to form fibers at the interface between the nutrient medium and air. The result is a three-dimensionally interwoven fiber network consisting of around 99% water and 1% nanocellulose.

The templates 31 can also be punching or cutting molds 31. Using cutting and milling tools (knives, scissors, punches, lasers, perforated pipes) or 3D printing processes, shapes can also be modeled from a solidly grown block or a thin membrane of a hydrogel that would not be produced by purely organic growth. This results in a large number of additional components in the modular system. The nonwoven punched or cut from the hydrogel using the punching or cutting mold 31 has the same shape as a nonwoven cultivated from hydrogel using the corresponding template 31.

A possible embodiment of a utensil 1 according to the invention is shown in FIG. 2. The utensil 1 is a wash and massage glove designed as a mitten. The utensil 1 has a first utensil element 10 and a second utensil element 20 (FIG. 2a). In this embodiment example, both utensil elements 10, 20 are made of a hydrogel. It is also possible that only one utensil element 10 is made of a hydrogel, the second utensil element can be made of a cotton fabric, for example. Both utensil elements 10, 20 are placed in two layers (FIG. 2b, c) and sewn and stitched or pressed along the seams 25.

FIG. 3 shows a further possible embodiment of a utensil 1 according to the invention. The utensil 1 is also a glove, which is designed as a moisturizing glove with a finger shape. The utensil 1 has a first utensil element 10 and a second utensil element 20 (FIG. 3a, b). In this embodiment example, both utensil elements 10, 20 are made of a hydrogel and cut to size. Both utensil elements 10, 20 are also placed in two layers and stitched along the seams 25 and crimped or pressed.

Further embodiments of utensils 1 laid in two layers and sewn and crimped or pressed together are shown in FIG. 4 and FIG. 5. The utensils 1 also each have a first utensil element 10 and a second utensil element 20. When brought together, the utensils 1 have a washcloth or tubular shape. The second utensil element 20 is preferably sewn with waterproof, slightly elastic polyester or nylon yarns 25. Sewing is possible both in the never-dried state of the nanocellulose and in dry form for subsequent swelling.

The hydrogels 100 made of microbial polymers, which are produced and purified in different ways, are preferably used in the following applications according to the invention and can be marketed individually, in a container or in a modular system: As a wet wipe or protective film, the nonwoven has a drained weight of 10-270 g with a size of 120-300 mm×120-300 mm and a thickness of 0.1-3 mm. The washcloth or massage sponge has a draining weight of 60-250 g with a size of 120-180 mm×120-180 mm and a thickness of 3-10 mm. Both formats (with rounded corners, round, oval or grown or cut at right angles) can be combined with a glass or plastic cylinder, which can be filled with warm water or skin care products.

FIG. 6 shows an embodiment example of a cultivation vessel 30 (FIG. 6a) with suitably shaped in-situ templates 31 (FIGS. 6b, 6c) for the production of a first utensil element 10 and/or second utensil element 2l from hydrogel. The cultivation vessel 30 is preferably made of glass or a food-safe plastic. The templates 31 correspond to the templates 31 from the first embodiment example (see FIG. 1). The templates 31 can have different shapes for producing a first utensil element 10 and/or a second utensil element 20. Using the template 31 shown in FIG. 6b, a first utensil element 10 and a second utensil element 20 are produced in the shape of a glove. The template shown in FIG. 6 c is used to produce circular utensil elements 10, 20, e.g. for nipple protectors. The dimensions of the templates 31 are such that the templates 31 can be arranged parallel to the base surface of the cultivation vessel 30 in the cultivation vessel 30.

The template 31 can specify the final shape of a utensil element 10, 20 or individual components for a specific design of more complex models in a modular system. Appropriate devices can also be used to design cylinders, tubular covers, limbs and components using the method described. The advantage over punching and cutting processes using knives, shears or lasers is the avoidance of waste and therefore efficient utilization of the raw materials used.

FIG. 7 shows an example of the production of a utensil element 10, 20. In particular, purified bacterial nanocellulose produced by various methods, such as the well-known Hestrin-Schramm method, on the basis of coconut, acetic acid with Gluconacetobacter xylinus, cynobacteria (“blue-green algae”), as well as a variant from cannabis tea and kombucha culture, is used here. The synthesis of bacterial nanocellulose can be organized with different raw materials, especially different carbon sources in the fermentation and thus be adapted to the local availability of resources.

Nanocellulose is preferably synthesized by cultivating microbial strains such as Gluconacetobacter, Enterobacter, Agrobacterium, Pseudomonas, and Rhizpobium. The bacteria are cultivated in the nutrient solution 33 in the cultivation vessel 30, supplemented if necessary with further active substances or nutrients 34 and can be stored in the container 50 for later inoculation of a culture medium. They can be mixed with nutrients and additives in water and thus initiate a new fermentation.

Alternatively or additionally, shapes can also be punched or milled from the thin membrane of the BNC nonwoven produced in this way with the aid of punching or cutting dies 31 using cutting and milling tools. The nonwoven then has a shape corresponding to the punched or cut shapes 31.

In addition to Gluctonacetobacter hansenii and Gluconacetobacter kombuchae, the bacterial strain Gluconacetobacter xylinus, which has also been most extensively researched and documented in this context, is particularly suitable for the production of dimensionally stable nanocellulose gels. The hydrogel 100 is produced by microorganisms at the interface between air and a nutrient medium containing sugar. The bacteria expel the cellulose as fibrils, which aggregate to form fibers at the interface between the nutrient medium and the air. The result is a three-dimensionally interwoven fiber network consisting of around 99% water and 1% nanocellulose. The shape of the utensil element 10, 20 is determined by the template 31 and its openings 32.

In contrast to alkaline cleansing or loading by means of an inoculation substance 33 with active ingredients 34 (such as aloe vera, hyaluronic acid, black tea), acidic loading with probiotic bacterial cultures may be desirable and give the hydrogel 100 special properties, for example in the treatment of certain fungal diseases (with lactic acid and lactic acid bacteria), psoriasis or as a carrier of a specific bacterial culture for the cultivation of bacterial nanocellulose.

From a chemical point of view, bacterially synthesized nanocellulose (BNC) is identical to plant cellulose, but differs significantly due to the specific biosynthesis process. BNC typically has a fiber diameter of around 40-60 nm, which corresponds to one hundredth of the fiber diameter of a plant cellulose or cotton fiber. Depending on the manufacturing process, fiber diameters can also be in the range of 20-100 nm. The nonwovens and utensils 1 produced according to the method of the invention have fibers with a diameter of 10 nm to 150 nm, preferably a diameter of 25 nm to 90 nm, particularly preferably a diameter of 50 to 70 nm. The fibers of the nonwovens and utensils 1 produced according to the method of the invention have a length of 100 μm to 1 mm, the density of the nonwoven is 1-1.5 g/cm³. Typically, the fiber length of the nonwovens is 5 μm to 15 mm.

Washing with sodium hydroxide solution in a concentration and duration adapted to the strength and composition of the hydrogel 100 makes the cell structure of the hydrogel 100 more flexible, softer and more pliable without significantly reducing its stability and water retention capacity for its intended purpose. NaOH can be used in varying concentrations (0.1 molar to 1 molar) to inactivate the bacterial strains and to wash out entoxins and other foreign substances from the culture medium produced during fermentation.

Inclusions and unwanted bacteria can be washed out in this way to ensure that the Hydrogel 100 is free of germs and impurities before it is used again. The pH value of a Hydrogel 100 can thus be influenced and adjusted to a desirable range for the skin.

The manufacturing method of the utensil element 10, 20 further optionally includes the following steps: dissolving, in a flat-bottomed vessel, crystalline glucose having a concentration of from 2% to 20% by weight, sodium hydrogen phosphate and citric acid in water to form a buffered pH between pH 4 to pH 7, introducing a dry mixture of peptone and a yeast extract each having a concentration between 0.1% and 5% by weight into the buffered aqueous solution, stirring the solution until the peptone is completely dissolved, adding a dry mixture of peptone and a yeast extract each having a concentration between 0.1% and 5% by weight to the buffered aqueous solution, stirring the solution until the peptone is completely dissolved and adding a dry mixture of peptone and a yeast extract each having a concentration between 0.1% and 5% by weight to the buffered aqueous solution % and 5% by weight into the buffered, aqueous solution, stirring the solution until the peptone has completely dissolved and the yeast extract is fully suspended. Sterilization by autoclaving at 121° C. for 20 minutes. Inoculation with bacterial strain Gluconacetobacter xylinus, cultivation of the solution for between 2 days and 25 days until a hydrogel 100 forms at the interface between the culture medium and the air, decanting of the aqueous solution, washing of the hydrogel, purification and subsequent optional soaking of the hydrogel 100 in an aqueous solution containing colouring agents, flavourings, fragrances and active ingredients for between 30 minutes and 30 days, and finally sterilization by superheated steam or e-beam.

FIG. 8 shows an example of a cylindrical BNC object. The BNC object has the hydrogel 100, is rolled (FIG. 8 a) and is preferably held in the cylindrical shape by means of an adapter 99 in the form of an elastic ring (FIG. 8 b) or two rubber bands (FIG. 8c). The inner and outer diameter of the BNC object can be varied by means of the adapter 99.

This BNC object is used for haptic stimulation and can be provided with sliding agents, e.g. a lubricant. Oily, greasy, fat-free and/or silicone-containing substances can be used as sliding agents. Glycerine, which is inexpensive and available, is particularly preferred. At the same time, it is harmless to the body and is widely used as an ingredient in creams, for example.

As a material unit for individual further processing by the user, the nonwoven 100 has a length of 150-400 mm and a width of 120-300 mm in a non-rolled, non-folded state. In rolled form, this results in a cylindrical shape of the stated length and, depending on the thickness of the material (0.5-25 mm), an outer diameter of 30-120 mm. The drained weight is 100-1200 g. Sizes vary, as the products are both industrially manufactured and can be customized to the needs of consumers.

FIG. 9 shows an example of the packaging for storing a cylindrical BNC object and its installation as a sheath. The hydrogel 100 is rolled (FIG. 9a) and is held by means of an adapter 99, here an elastic ring or rubber band 99 (FIG. 9c) in a non-packaged state on the stabilizing cylindrical vessel shape in order to encase it and form a solid object with an outer skin of nanocellulose. The packaging of the biopolymers in the never-dried state should be vacuum-packed in a water-impermeable film, without air supply in liquid (distilled water or in loading solution) or, in the interests of sustainability, in a reusable cylindrical container 50 that can be closed with the screw cap 51.

The high water content of up to 99.5% is of particular importance for all applications and the nanocellulose can naturally only be reused for these purposes if it remains hydrated and does not dry out without appropriate preparation. Therefore, storage, loading and renovation take place in liquid, preferably in water or in an aqueous solution.

The airtight and watertight container 50 is the most sustainable option for storing, cleaning and loading the products, particularly in the case of hydrogels 100, whose safe reusability depends heavily on proper handling and compliance with hygiene requirements. The combination possibilities with nanocellulose hydrogels can be illustrated using the example of a screw-top jar or a watertight sealable cylinder. In this case, the screw thread makes it possible to fix a rolled hydrogel 100 protruding into the container with a rubber band or ring 99 in order to obtain a hollow body open on one side with a BNC inlay in just a few simple steps. This can be extended as required by repeating the process with further hydrogels 100 of the desired thickness, for example to adapt the inside diameter of the opening 13 or the contents of the container 50 to individual requirements. The utensil 1 can thus take on very different shapes in detail, as the contents, opening and sheathing can be modeled according to individual wishes. The circumference and length can be selected to suit every anatomical feature and application. Diameters of 1-10 cm and lengths of 5-30 cm of Container 50 and its wide-necked opening can be combined with hydrogel shapes with 5-40 cm long outer sides (rectangular) or diameters (round). The assembled object can also be filled with hot water, other functional materials or objects (sponge, stimulation utensils) and can also be used to absorb liquids. All components are in principle reusable, washable and can be hygienically cleaned with soap or disinfectant, boiled or sterilized before being used again.

FIG. 10, FIG. 11 and FIG. 12 show embodiments of containers 50 as well as the arrangement of cylindrical BNC objects on or in the container 50. The corresponding container 50, a cylindrical glass (also acrylic glass or food-safe plastics) with a screw cap or snap-on cap, cork or lid made of plastic or aluminum and at least one wide-necked opening, represents the container 50 in which the BNC object is regenerated and/or loaded, stored and/or cleaned.

The screw-top jar 50 can be either clearly transparent or completely opaque-amber glass, for example, represents an intermediate form-but the container 50 can also be a liquid-tight, resealable form made of silicone. An object produced using a 3D printing process for appropriate use with a hydrogel 100 also falls within the possible combinations of the invention.

In these embodiments, the container 50 is a cylinder that is open on both sides. As an alternative to the previous embodiment example (see FIG. 9), a hydrogel 100 is wrapped around the container 50 as a sheath and fixed with rubber bands 99 or cuffs 99 and tucked into the container 50 in order to achieve certain effects of air pressure and vacuum. These include the manual control of the vacuum, the function of an adapter 99 (for stabilizing or wrapping a body part) or the simultaneous use of several users. A simplified version with BNC inner and outer surfaces is created by rolling up a hydrogel 100 (8a) and fixing it with one or more rings, rubbers or cuffs 99 or shaping it with another tubular cover, which can also be a commercially available condom if a secure closure to one side is desired. In addition, other desired hydrogels 100 or utensils 1 can be fixed in place.

The cylindrical container 50 serves as a further example for the storage and encasing of objects, but can also represent any shape and commercially available massage or stimulation utensil with which a dimensionally stable hydrogel 100 can be combined in order to avoid direct contact with the skin, to create a novel haptic or visual experience or to give them cooling or warming properties with the aid of the high water content of the hydrogel 100 as a temperature reservoir. These include commercially available massage devices such as pimple balls, massage wands, stimulation utensils with or without vibration, including replicas and simulations of certain bodies or body parts. The dimensionally stable Hydrogel 100 also takes on the function of a sliding agent here, is reusable and can therefore reduce the consumption of water-or silicone-based sliding agents and, if used correctly, help to reduce packaging waste. An efficient BNC-containing sliding agent gel for single use is also feasible, which has BNC particles instead of dimensionally stable nonwovens by mechanically crushing them beforehand or producing them directly in small pieces by generating a movement of the culture medium during cultivation to prevent the fibers from forming into a nonwoven.

Various substances for loading can be supplied as an option and complete a set, such as a modular system of active ingredients, colorants and fragrances for the individual composition of do-it-yourself cosmetics or an antiviral, antibacterial solution of disinfectants and additives. The airtight and watertight Container 50 is also the most sustainable option for packaging, storing, cleaning and loading the products for hydrogels 100, whose safe reusability depends heavily on proper handling and compliance with hygiene requirements.

The hydrogel 100 in hydrogenated or dried form can be combined according to the intended use and thus also be offered in the form of a set or modular system with other hardware (vessels, connecting and fixing elements), tools, toys and ingredients. In addition to ingredients for loading, these also include those that simplify the domestic or industrial production of BNC nonwovens, such as an instant powder with nutrients or particularly suitable bacterial cultures and yeasts. A modular system for experimenting with and producing bacterial nanocellulose for education and teaching, molecular cuisine or home use is not yet known. The use of the hydrogels described here in body and physiotherapy or in immersive simulations (e.g. also for training purposes in dermatology, surgery or in augmented and virtual reality scenarios and future educational, entertainment and knowledge transfer offers) has not yet been documented.

In order to minimize transport costs and packaging, the hydrogels 100 can be pre-treated with a (3% by weight) glycerine solution ex works or for home use (inlaid) and achieve a weight reduction to 3-15% of the original hydrated form using various known drying processes (e.g. air drying, spray drying, freeze drying, oven drying). When re-swelling in water or an aqueous solution, the hydrogel absorbs up to 90% of the original amount of liquid again and is able to bind this liquid thanks to its nanofiber network with high water retention capacity. A further advantage of the dried nonwovens is that additives are absorbed more efficiently by the swelling hydrogel 100 than in the case of an already swollen hydrogel. Furthermore, the glycerine released from the fiber network during swelling can serve as a sliding agent.

Due to the different formats and thicknesses of the hydrogels 100 and fluctuating volumes due to their swelling (also production-related), small vessels 50 from 10 ml up to large cylinders with a capacity of 3 liters and diameters (or long sides in the case of rectangular vessels 50) of up to 15 cm are used, which can be up to 30 cm high with correspondingly smaller base areas.

The sizes vary, as the products can be industrially manufactured, individually adapted to the needs of consumers or manufactured and designed by so-called prosumers or business customers themselves with the help of an extended modular system. The modular system supplements the present invention with ingredients and devices that can be used for the independent production, processing, loading and care of the hydrogels.

FIG. 13 shows a top view of embodiments of cylindrical BNC objects with different, individually adapted openings. In addition to preventing a viral or bacterial infection, the hydrogel 100 can also intensify haptic and visual perception, be used as a toy, haptic stimulant and sliding agent or provide protection against mechanical overstress. These nanocellulose components can be joined together using adapters 99 such as rubber bands, cords, cuffs, rings, clamps, clips or the technique of sewing. Massage tools, bags, containers such as jars, bottles or tubes can be combined in a shaping and stabilizing way or used for storage.

Hydrogels 100 can therefore be used in conjunction with massage devices and rods, vibrators, rings, as a freely customizable temporary prosthesis or in combination with appropriate devices. These can also include vibrators or devices that generate a vacuum or simulate muscle contractions or movements. More complex combinations with robotics, sensors and wireless technologies can be used in the field of mixed, virtual and augmented reality.

Sex aids and stimulation devices usually aim to stimulate only a specific part of the body or to simulate specific body parts. These are often geared towards either the male or female anatomy, gender identity and preference, leaving limited scope for imaginative use and a holistic, multi-sensory experience. In combination with cylindrical objects, vessels, rings or massage rods, a hydrogel can therefore be used to form an outer skin, inner skin, sheath, erectile tissue or body part, in individually selectable dimensions and scales. This can be used to achieve a specific haptic or visual experience or to prevent direct skin contact with objects.

Already in use as a carrier system in cosmetics (active ingredient-soaked cloth masks, face pads) and in medicine (wound dressings, wound plasters) as disposable products in mostly aluminium packaging, the novel applications of bacterial nanocellulose are now aimed at reusability, the reduction of packaging waste and transport routes, customizable application scenarios (DIY cosmetics) and household (hygiene, wet wipes, disinfectant wipes, medical self-care). The use of non-allergenic, organic, biodegradable polymers as an alternative to certain latex or petroleum-based products can be made resource-saving and climate-neutral with this technology and offers the greatest possible compatibility for allergy sufferers and sensitive skin types.

The method of manufacture further optionally includes the following steps: dissolving, in a flat-bottomed vessel, crystalline glucose having a concentration of from 2% to 20% by weight, sodium hydrogen phosphate and citric acid in water to form a buffered pH between pH 4 to pH 7, introducing a dry mixture of peptone and a yeast extract each having a concentration of between 0.1% and 5% by weight into the buffered aqueous solution, stirring the solution until the peptone is completely dissolved and the yeast extract is completely suspended, stirring the solution until the peptone is completely dissolved and the yeast extract is completely suspended % and 5% by weight into the buffered, aqueous solution, stirring the solution until the peptone has completely dissolved and the yeast extract is fully suspended. Sterilization by autoclaving at 121° C. for 20 minutes. Inoculation with bacterial strain Gluconacetobacter xylinus, cultivation of the solution for between 2 days and 25 days until a hydrogel forms at the interface between the culture medium and the air, decanting of the aqueous solution, washing of the hydrogel, purification and subsequent optional soaking of the hydrogel in an aqueous solution containing colouring agents, flavourings, fragrances and active ingredients for between 30 minutes and 30 days, and finally sterilization by superheated steam.

In an alternative method, instead of introducing glucose, peptone, yeast, sodium hydrogen phosphate and citric acid, the following step is carried out: Incorporating a powder comprising 2% to 10% by weight of an extract of black tea, green tea and/or cannabis tea, 90% to 98% by weight of sucrose and/or glucose, 1% to 5% by weight of fruit or vegetable powder, dried leaves and flowers and dried herbal flavors and active ingredients.

The inoculation is carried out by adding dried microbes or a liquid solution containing them, at least one species selected from the group comprising: Gluconacetobacter xylinus, Gluconacetobacter kombuchae, Komagataeibacter hansenii, Gluconobacter oxydans, Saccharomyces ludwigii, Saccharomyces apiculatus or Saccharomyces cerevisiae. In addition, at least one further organic acid with a concentration of 0.1% by weight to 5% by weight is added, selected from the group comprising: gluconic acid, glucuronic acid, dextrorotatory (L+) lactic acid, tartaric acid, folic acid, oxalic acid, usnic acid, succinic acid, malic acid, malonic acid and citric acid. The sum of the weight percentages of the ingredients together is 100% by weight.

Alternatively, instead of adding glucose, peptone, yeast, sodium hydrogen phosphate and citric acid, the following step can be carried out: Introduce a solution of 300 g of white refined beet sugar or cane sugar in 2 l of coconut water, and 120 ml of concentrated anhydrous acetic acid.

In a further embodiment of the method, instead of introducing glucose, peptone, yeast, sodium hydrogen phosphate and citric acid, and instead of inoculation with Komagataeibacter xylinus, the following steps are carried out: Introduction of a solution of 5 g of dried cannabis flowers or leaves boiled in 1000 ml of water with the addition of a teaspoon of coconut oil for 60 min, mixed with 100 g of sugar (white refined beet sugar or cane sugar), cooled to room temperature and introduction of 250 ml of acidic kombucha tea (pH 2.2-pH 3.5) containing an active kombucha culture (such as live Gluconacetobacter kombuchae).

A kit for the use of any of the aforementioned manufacturing methods comprises 2% to 10% by weight of an extract of black tea, green tea and/or cannabis, 90% to 98% by weight of sucrose and/or glucose, 1% to 5% by weight of fruit or vegetable powder, dried leaves and flowers, dried herbal flavors and active ingredients, and dried microbes of at least one species selected from the group consisting of: Gluconacetobacter xylinus, Gluconacetobacter kombuchae, Komagataeibacter hansenii, Gluconobacter oxydans, Saccharomyces ludwigii,

Saccharomyces apiculatus or Saccharomyces cerevisiae.

The set also comprises at least one further organic acid with a concentration of 0.1% by weight to 5% by weight selected from the group comprising: acetic acid, gluconic acid, glucuronic acid, dextrorotatory (L+) lactic acid, tartaric acid, folic acid, oxalic acid, usnic acid, succinic acid, malic acid, malonic acid and citric acid. The sum of the weight percentages of the components together is 100% by weight. The set is composed in such a way that a pH value of 3.5 to 7 is formed in aqueous solution.

The process for producing bacterial nanocellulose with a dry instant mixture or the described two-component solution is new. Key production parameters are standardized here, which leads to a greatly simplified and more predictable result. This process can also be used in the food sector (e.g. Kombucha drinks) or textiles (production of vegan leather or fabrics based on bacterial nanocellulose), as the process step of brewing and cooling the tea is no longer necessary and the mixing ratio between the ingredients remains constant. The instant blend is a great simplification, especially for home users.

Kombucha, with its traditional home brewing and fermentation culture, often appears as an undefined culture, but usually contains a desirable probiotic composition of bacteria and yeast strains. However, this can vary greatly due to the nature of wild fermentation. Known components included here are: Gluconoacetobacter xylinus, Gluconoacetobacter kombuchae, Gluconoacetobacter hansenii Acetobacter xylinoides, Gluconobacter oxydans, Saccharomyces ludwigii, Saccharomyces apiculatus, Saccharomyces cerevisiae.

Organic acids include acetic acid, gluconic acid, glucuronic acid, dextrorotatory (L+) lactic acid, tartaric acid, folic acid, oxalic acid, usnic acid, traces of succinic acid, malic acid, malonic acid and citric acid. Trace elements and minerals include iron, magnesium, sodium, potassium, calcium, copper, zinc, manganese, cobalt and other minerals.

The list of vitamins includes vitamin B1, vitamin B2, vitamin B3, vitamin B6, vitamin B12, vitamin C, vitamin D, vitamin E, vitamin K. It also contains various amino acids, enzymes, tannins, the enzymes invertase, amylase, catalase, saccharase, rennet ferment and a proteolytic ferment, antibiotic substances, alcohol and carbonic acid.

The bacterial and yeast cultures of Kombucha have the special property of being able to assert themselves in sufficiently acidic liquid against foreign bacteria that threaten the system. If they are supplied with nutrients, natural colorants, active ingredients and flavors, the growing bacterial cellulose with its high water absorption (approx. 99% water, 1% cellulose) and its water retention capacity takes on additional properties.

Active nanocellulose gels contain live probiotic bacterial strains, in passive nanocellulose gels the bacterial strains were killed and extracted by purification process steps, and the hydrogels were sterilized by autoclaving (121° C. steam for 15-20 minutes) or electron beam process.

The combination of 250 ml of acidic Kombucha tea [“Fairment Kombucha-Original” pH 2.5-2.8] or a defined nanocellulose gel strain (pH 2.2-3.5) with a suitable active bacterial culture and 25 g of instant powder is suitable for producing one or more Kombucha-based nanocellulose gels with a total mass of 50 g or more in 2-25 days of standing cultivation under hygienic conditions and oxygen supply. The material properties of the kombucha-based nanocellulose gels are similar to the synthesized biopolymers mentioned above.

The liquid by-product is an acidic tea solution with a standard Kombucha composition of bacterial and yeast cultures (pH 2.3-4) with proportions of organic aroma, color, flavor and active ingredients (from fruit or vegetables, teas, plant aromas and active ingredients according to the composition of the instant powder). This solution is now suitable for inoculating and coloring, as well as for storing, renovating and maintaining a nanocellulose gel. It can also be used to revitalize a sterilized, passive nonwoven with the probiotic culture.

Due to the high water absorption and the ability to release water under mechanical influences, weight specifications are subject to strong fluctuations and can only be used here as a guide. Growth is also decisively influenced by the container. The shape, surface, fill level and material are factors that determine the properties. Porcelain, plastic and glass containers are best suited for growing nanocellulose at home.

In addition to synthesized, passive nanocellulose nonwovens and the probiotically active hydrogels, there is a third method of producing a nanocellulose gel based on coconut, which in turn has similar material properties to the above-mentioned nanocellulose gels and can also be loaded in situ or subsequently. Here, too, the production process of fermentation in stand cultivation over 2 days to 25 days is suitable, whereby the nutrient medium (pH 2.3-3.5) is composed as follows: 120 ml concentrated, anhydrous acetic acid (glacial acetic acid), 300 g sugar [Naturata organic beet sugar] (white, refined, alternatively raw cane sugar), 300 ml Nata starter (bacterial strain Gluconacetobacter xylinus), alternatively a Kombucha starter or non-pasteurized Kombucha drink [“Fairment Kombucha-Original”] can be used, also 2 l coconut water [“Coco Juice pure organic”].

A fourth way to produce bacterial nanocellulose is to use medicinally active cannabis. The nutrient solution is prepared by boiling 5 g of cannabis leaves or flowers with 1 l of water and a teaspoon of coconut oil for 60 minutes and adding 100 g of sugar [Naturata organic beet sugar] (white, refined, alternatively raw cane sugar). The addition of 250 ml of acidic Kombucha tea [Fairment Kombucha-Original pH 2.5-2.8] or a defined nanocellulose gel strain (pH 2.2-3.5) triggers the production of nanocellulose, which contains active cannabinoids in addition to the properties mentioned above. There are many receptors for cannabinoids in the skin and mucous membranes. The medicinally active ingredient cannabidiol (CBD), for example, promotes blood circulation in the tissue, which can lead to increased sensitivity.

The hydrogels produced in different ways are preferably used in the following product variants: The wet wipe or protective film has a drained weight of 12-180 g with a size of 150-300 mm×150-300 mm and a thickness of 0.1-3 mm. The washcloth (or massage sponge or haptic stimulant) has a draining weight of 60-180 g with a size of 120-180 mm×120-180 mm and a thickness of 3-10 mm.

Both formats (with rounded corners or cut at right angles) can be combined with a glass or plastic cylinder. A wash mitt results from the combination of two cloths (e.g. square, rectangular, in the shape of a hand, etc.), which can be cut, sewn and crimped or pressed in a similar way to textiles.

As a material unit for individual further processing, the nonwoven has a length of 150-400 mm and a width of 120-300 mm when not rolled. In rolled form, this results in a cylindrical shape of the stated length and, depending on the thickness of the material (0.5-25 mm), an outer diameter of 30-120 mm. The drained weight is 100-1200 g. Sizes vary, as the products are both industrially manufactured and can be individually adapted to the needs of consumers. By growing them in a suitably shaped container-preferably made of glass or a food-safe plastic-foldable and rollable flaps (such as circular, oval, rectangular, square, diamond-shaped, triangular) can be produced with variable thickness/thickness, depending on the duration of fermentation, temperature and the addition of nutrients. Using appropriate devices and vessels, the process described can also be used to create various components such as cylinders, tubular coatings and protectors.

Using cutting and milling tools (knives, shears, punches, lasers, perforated pipes) or 3D printing processes, shapes can be modeled from a solidly grown block or a thin membrane that would not be created by purely organic growth. This results in a large number of other components in the modular system.

These nanocellulose gel components can be joined together using adapters 99 such as rubber bands, cords, cuffs, rings, clamps, clips or the technique of sewing. Massage tools, bags, containers such as jars, bottles or tubes can be combined in a shaping and stabilizing way or used for storage.

Tools such as templates made of plastic, glass or cork can also be used in the rearing process to shape the bacterial nanocellulose gel growing on the surface while it is still growing. This can represent the final shape or individual components for a specific design of more complex nanocellulose gel-based models. Lasers, embossing dies, embossing tools and branding dies can be used to mark or design objects made of bacterial nanocellulose with model numbers, manufacturing data or other information.

The ecological opportunity of spreading the technology in skin applications is the reduction of plastic waste and the use of non-renewable raw materials. The possibility of resource-conserving individual domestic production, as well as the possibility of choosing from various manufacturing processes in order to be able to access regionally produced and easily available raw materials on an industrial scale, means that packaging, delivery routes and thus emissions can be avoided.

The ideal duration of purification following cultivation and the concentration of NaOH (in distilled water) depends on the exact composition of nutrients and bacteria used, whether they are composites, hybrids or pure cultures.

In one embodiment of the process according to the invention, a one-step cleaning process is used. For each millimeter of thickness of the nonwoven, a reliable termination of all bacterial activity is achieved with a reaction time of 2 hours at 85° C. in 100 ml of 0.8 wt. % NaOH solution per cubic centimeter of cellulose in all the processes mentioned. Depending on the desired degree of purity, this process step can be repeated with the renewal of the sodium hydroxide solution as often as necessary-or in a dynamic cycle-until this NaOH solution absorbs no or only minor detectable impurities from the cellulose produced. The nonwoven is then rinsed with distilled water and, depending on the intended use, the pH is adjusted to the desired value between pH 4 and pH 7-preferably to a skin-neutral value of about 7, so that the pH can be easily readjusted by subsequent appropriate loading. Optionally, citric acid can also be used in addition to distilled water for this neutralization step. The nonwoven cleaned in this way has an (intended) loading of 8% by weight and 1.5% by weight of impurities. Purification can optionally be completed with sterilization, for example by hot steam for 20 minutes at 121° C., in order to kill other microorganisms and ensure the longest possible shelf life in the packaging.

In a further embodiment of a one-step cleaning process, the nonwoven is cleaned at 110° C. for 240 minutes with a 45% by weight NaOH solution. The amount of NaOH solution is also 100 ml per cubic centimeter of cellulose. After cleaning, the nonwoven is also rinsed with distilled water and optionally citric acid. The nonwoven cleaned in this way has a loading of 9.5% by weight and 0.8% by weight of impurities.

In a further embodiment of the method according to the invention, a two-stage cleaning process is used. In the first stage (coarse cleaning), a NaOH solution with 50 wt. % NaOH is used, the duration of action is 135 min, the temperature 127° C. In the second cleaning stage, the nonwoven is cleaned with an 8% by weight NaOH solution at a temperature of 85° C. for 240 minutes. The web cleaned in this way has a loading of 10.5% by weight and 0.27% by weight of impurities.

### LIST OF REFERENCE SYMBOLS

1 Utensil

10 First utensil element

20 Second utensil element

30 Cultivation vessel

31 In-situ stencil

32 Base area of an in-situ template for cultivation

33 Nutrient solution containing bacteria

34 Nutrients/additives

40 Container

50 Vessel

51 Lid

60 Cylinder, open on both sides

99 Adapter

100 BNC nonwoven

1. A method of manufacturing a utensil (1) comprising a plurality of utensil elements (10, 20), comprising the following method steps:
Providing a first utensil element (10) wherein the first utensil element (10) is made of a hydrogel
Providing a second utensil element (20)
Providing a connecting element
Positive and/or non-positive connection of the first utensil element (10) to the second utensil element (20) with the aid of the connecting element.

2. A method of manufacturing a utensil (1) comprising a plurality of utensil elements (10, 20) according to claim 1
characterized in that the connecting element is a yarn, ring, clamp, clip, rubber, clip and/or rivet.

3. A method of manufacturing a utensil (1) comprising a plurality of utensil elements (10, 20) according to claim 1
characterized in that several connecting elements are used to connect the first (10) and the second utensil element (20).

4. A method of manufacturing a utensil (1) comprising a plurality of utensil elements (10, 20) according to claim 3
characterized in that different connecting elements are used to connect the first (10) and the second utensil element (20).

5. A method of manufacturing a utensil (1) comprising a plurality of utensil elements (10, 20) according to claim 1
characterized in that
the utensil elements (10, 20) produced from a hydrogel are produced by a process comprising the steps of providing and inoculating the sugar-containing solution with a bacterial strain and cultivating the solution.

6. A method of manufacturing a utensil (1) comprising a plurality of utensil elements (10, 20) according to claim 5
characterized in that the hydrogel resulting from the cultivation is washed.

7. A method of manufacturing a utensil (1) comprising a plurality of utensil elements (10, 20) according to claim 6
characterized in that the hydrogel resulting from the cultivation is washed in a caustic solution containing 5 wt. % to 50 wt. % at 37°° C. to 142° C. for 5 min to 400 min, the caustic solution being a 5 wt. % to 50 wt. % sodium hydroxide solution.

8. Process for the preparation of a utensil (1) consisting of bacterial nanocellulose according to claim 6
characterized in that sterilization is carried out after the washing process.

9. A method of manufacturing a utensil (1) comprising a plurality of utensil elements (10, 20) according to claim 1
characterized in that the utensil (1) is a stimulation utensil.

10. Nonwoven made from a hydrogel (100) of bacterial nanocellulose with a cellulose content of more than 0.5% by weight
characterized in that the nonwoven is loaded with a sliding agent.

11. Nonwoven made of a hydrogel (100) of bacterial nanocellulose according to claim 10
characterized in that the hydrogel (100) has a water content of between 80% by weight and 99.5% by weight.

12. A nonwoven made of a hydrogel (100) of bacterial nanocellulose according to claim 10
characterized in that the sliding agent is a lubricant.

13. A nonwoven comprising a hydrogel (100) of bacterial nanocellulose according to claim 12
characterized in that the sliding agent is an oily, greasy, fat-free and/or silicone-containing substance.

14. A nonwoven comprising a hydrogel (100) of bacterial nanocellulose according to claim 12
characterized in that the sliding agent is glycerine.

15. A nonwoven web of a hydrogel (100) of bacterial nanocellulose according to claim 10
characterized in that the sliding agent in the nonwoven is present as an aqueous emulsion.

16. A nonwoven web of a hydrogel (100) of bacterial nanocellulose according to claim 10
characterized in that the nonwoven is produced according to a method comprising:
Providing a first utensil element (10) wherein the first utensil element (10) is made of a hydrogel
Providing a second utensil element (20)
Providing a connecting element
Positive and/or non-positive connection of the first utensil element (10) to the second utensil element (20) with the aid of the connecting element.

17.-23. (canceled)
